(19) United States
(12) Patent Application Publication
(10) Pub. No.: US 2026/0340871 A1
(43) Pub. Date: Oct. 29, 2026

(54) DEVICE AND COMPUTER-IMPLEMENTED METHOD FOR CALIBRATING A SYSTEM

(71) Applicant: VOLUME GRAPHICS GMBH, Heidelberg (DE)

(72) Inventor: Florian Cornelius RIEGER, Heidelberg (DE)

(73) Assignee: VOLUME GRAPHICS GMBH, Heidelberg (DE)

(21) Appl. No.: 19/116,204

(22) PCT Filed: Sep. 27, 2023
(86) PCT No.: PCT/EP2023/076669

§ 371 Date: Mar. 27, 2025

(30) Foreign Application Priority Data
Sep. 27, 2022 (DE) 10 2022 124 782.1

(51) Int. Cl. G06T 7/80; G06T 7/70

(52) U.S. Cl. CPC G06T 7/80; G06T 7/70

(57) ABSTRACT

The disclosure relates to a device for calibrating a system for generating an image of an object, where the device has a multiplicity of calibration objects and a base body with corners and outer edges that extend between the corners and delimit surface elements. In some embodiments, a first subset of the calibration objects is arranged on the corners of the base body, where a second subset of the calibration objects is arranged on the outer edges between the corners and a third subset of the calibration objects is arranged between the outer edges on the surface elements. The disclosure may additionally include a computer-implemented method that uses the device. Embodiments herein thus relate to a device and a method for calibrating a system for generating at least one image of an object that can be used to determine detector parameters with high accuracy with comparatively little computing complexity.

The invention relates to a device and a computer-implemented method for calibrating a system for generating at least one image of an object.

Systems for generating at least one image of an object can be used in different fields. Taking the example of workpiece testing, manufactured workpieces can be examined using optical methods, for example, in order to compare the surfaces of the manufactured workpiece with target parameters. Furthermore, radiographic measurements, e.g. computed tomography measurements, can be used for the examination, e.g. dimensional measurements, of the manufactured workpiece.

Performance of the examinations requires calibration of the imaging system. Especially for computed tomography scans and in particular for robot CT, it is necessary to know the geometric calibration. In the case of a computed tomography system for generating images of an object, geometric calibration concerns the geometric positioning and orientation of the components radiation source, detector and measurement object in relation to one another for each individual recorded radiograph. The geometric positioning and orientation of the components can also be referred to as the recording geometry. Furthermore, the individual recorded radiograph can be referred to as a projection or projection image. In the case of optical systems, the recording geometry concerns the positioning and orientation of the camera and the object in relation to one another.

In order to be able to perform a computed tomography reconstruction on the basis of images, in particular radiographs, it is necessary for as accurate as possible a quantitative description of the recording geometry to be available for each individual image. Especially with radiographs, errors in the geometric calibration cause image errors in the reconstructed volume data, e.g. blurred edges, double edges, distortions, and thus measurement errors when performing dimensional measurements or other analyses, for example defect or pore and porosity analysis. Geometric calibration can also be relevant to an evaluation of radiographs without tomographic reconstruction, e.g. when multiple radiographs are evaluated together in order to determine the position in space for a recognized property or a recognized feature of the mapped object as a result of viewing it from different directions. Geometric calibration errors also cause measurement errors here.

A quantitative description of the recording geometry for a radiograph is provided e.g. by defining the nine parameters of a pinhole camera. If, in addition to a Euclidean camera reference system with the radiation source at the coordinate origin and the detector plane parallel to one of the coordinate planes, a Euclidean object reference system that is fixed for all recordings but arbitrary is selected, three so-called intrinsic camera parameters describe the mapping of three-dimensional coordinates of the camera reference system to the image coordinate system. The remaining six so-called extrinsic camera parameters describe the relative position and orientation of the camera and the object reference system. Three of the six extrinsic camera parameters define a translation vector that describes the relative position of the camera and the object reference system. The other three extrinsic camera parameters define a rotation that describes a relative orientation of the camera and the object reference system. The sum total of the extrinsic camera parameters is referred to as a pose. The camera parameters can also be subsumed under the term detector parameters hereinbelow.

Defining the intrinsic and extrinsic camera parameters thus determines how each point of the object to be mapped, which is described by three-dimensional coordinates in the object reference system, is to be mapped to a pixel in the image or in the radiograph. The pose, which comprises six of the camera parameters, as explained above, describes the mapping of three-dimensional coordinates from the object reference system to three-dimensional coordinates in the camera reference system. The intrinsic camera parameters describe the mapping of these three-dimensional coordinates in the camera reference system to two-dimensional coordinates in the image, which can also be referred to as pixel coordinates.

In the general case, especially with complex trajectories in robot CT, there is a separate set of nine camera parameters to be determined for each individual radiograph. The reconstruction can then reconstruct the object volume for this set of radiographs of the measured object on the basis of the camera parameters, the camera parameters acting as calibration parameters.

It is known practice to perform a so-called offline calibration, wherein a dedicated phantom is mapped or irradiated in all required recording geometries in advance or retrospectively, i.e. for each image or for each radiograph of the object to be measured an image or a radiograph of the phantom is also recorded with preferably the same recording geometry. By analysing the image or radiographs of the phantom, the recording geometry for each image or radiograph of the object to be measured is determined or estimated.

The term “image” hereinbelow also means a radiograph.

U.S. Pat. No. 10,977,839B2 discloses a phantom in which individual spheres are arranged along a straight line. For each of four spheres, a numerical descriptor is calculated which is invariant even with mapping to a two-dimensional projection. The descriptor is therefore constant in every projection in which the spheres can be recognized. Therefore, correspondences to three-dimensional coordinates can be produced between the two-dimensional coordinates of the image. Furthermore, they can be used to estimate the intrinsic and extrinsic camera parameters. The estimated camera parameters can then be optimized to obtain accurate calibration.

The object of the invention is to provide a device and method for calibrating a system for generating at least one image of an object that can be used to determine the detector parameters with high accuracy with comparatively little computational effort.

Main features of the invention are specified in claims 1, 9 and 14. Claims 2 to 8 and 10 to 13 relate to refinements.

In a device for calibrating a system for generating at least one image of an object, wherein the device comprises a plurality of calibration objects and a base body having vertices and outer edges that extend between the vertices and delimit surface elements, wherein a first subset of the calibration objects is arranged on the vertices of the base body, the invention provides for a second subset of the calibration objects to be arranged on the outer edges between the vertices, and for a third subset of the calibration objects to be arranged between the outer edges on the surface elements.

The invention thus provides a device which can be used to determine an estimation of the detector parameters by way of the known relationships between the different subsets of the calibration objects and between the calibration objects between and within the subsets. In the case of a radiographic measurement, the calibration objects have absorption properties for the radiation of the radiographic measurement which differ from the absorption properties of the base body. Furthermore, the calibration objects are preferably in three-dimensional form. The calibration objects of the first subset, which are arranged on the vertices of the surface elements, specify parameters for the general shape of the surface elements. Extending between the vertices are preferably straight outer edges on which the calibration objects of the second subset are arranged. This means that the contours of the surface elements are represented by the first subset and the second subset of the calibration objects in an image in a manner that can be checked. Determination or estimation of detector parameters can therefore result in the mapped surface elements in at least one image of the device being determined by considering or using at least the known positional relationships between the calibration objects of the first and second subsets. If the surface elements are e.g. triangles, all triangles formed by three respective mapped calibration objects in the image can be examined for whether a further calibration object is arranged on the connecting line between two of the three mapped calibration objects, corresponding to the outer edge defined by the triangle. If this is not the case, the triangle checked in this way can be discarded. Otherwise, there is a high probability that a surface element of the device is being mapped. In order to decide whether a surface element is being mapped, it is possible to check whether the triangle has a calibration object of the third subset. If it is known where the calibration objects of the third subset are arranged in relation to the calibration objects of the other two subsets, this check can be simplified. Once the surface elements have been found, various test calibrations, so-called initial guesses, can then thereby be created one after the other, which transfer an e.g. calculated test mapping of the first and second subsets of the calibration objects of the device to the vertices or outer edges of the mapped device. The test calibration corresponds to a mapping instruction between the device and the mapped device. If a test calibration is not correct, the calibration objects of the third subset are not transferred to the correct positions in the surface elements. Only when the correct test calibration has been found will all calibration objects be mapped correctly. This test calibration can then be optimized further to obtain accurate calibration. The invention thus provides a device that can be used to determine the detector parameters with high accuracy with comparatively little computational effort.

According to one example, the arrangement of the calibration objects of the third subset relative to the calibration objects of the first subset and the second subset can differ between at least two surface elements.

In this example, the calibration objects of the third subset can encode a specific arrangement of the surface elements among one another. In particular, if all surface elements have the same shape or contour, a specific arrangement of the surface elements among one another can be identified by means of the third subset in an image of the device.

In another example, each outer edge can have at most one associated calibration object of the second subset of the calibration objects, and/or the second subset of the calibration objects can be arranged centrally between the vertices on each outer edge.

In this case, only at most one calibration object of the second subset can be arranged between calibration objects of the first subset. This allows the number of overlaps in a mapping of the calibration objects to be reduced or, ideally, avoided completely. It also becomes easier to determine the surface elements when the calibration objects of the second subset are arranged centrally between the vertices on the outer edges. This means that the calibration objects of the second subset are at the same distance, within a tolerance, from the two vertices of the outer edge on which the calibration objects are arranged.

Furthermore, the calibration objects can be arranged in at least two planes, for example, each plane having at least four of the plurality of the calibration objects, with preferably only part of the planes being parallel to one another.

Different planes can be defined e.g. by different surface elements. The surface elements can form different and/or opposite sides of the base body. The more calibration objects are arranged in a plane, the easier it becomes to identify the respective plane.

In another example, the planes can be non-parallel to one another. The surface elements with which the planes are associated may e.g. have a common outer edge at the cut edge of the planes. Furthermore, the base body may be in a form, for example, such that the surface elements of two opposite sides can be arranged so as to be non-parallel to one another.

According to another example, each surface element can have at least two, preferably at least three, more preferably at least four, predefined positions internally, wherein each surface element has an associated calibration object of the third subset of the calibration objects, which calibration object is arranged at one of the predefined positions and the other predefined positions, preferably at least two, more preferably at least three, more preferably at least four, of the other predefined positions, can be free of calibration objects, wherein more preferably as large as possible a subset of the predefined positions or even the entire set of the predefined positions can be invariant under all symmetry transformations of the surface element.

For example, four predefined positions can be the centers of four possibly equilateral, disjoint, with the exception of the edges, triangles whose union results in exactly the surface element.

The calibration objects can thus be effectively used for encoding the individual vertices of the surface elements. The distribution of the calibration objects of the third subset over the predefined positions can thus be in a form such that the arrangement of the calibration objects allows unique association of the surface elements. This means that each orientation of the device can have a unique distribution of the calibration objects of the third subset. This can result in more reliable determination of a correct test calibration.

In another example, the relative arrangement of the calibration objects of the first subset and the second subset in relation to one another on each surface element can be the same.

The homogeneous relative arrangement of the calibration objects of the first subset and the second subset in relation to one another can make it easier to identify the surface elements in the image. At the same time, the surface elements then all have the same shape and size, and so the identification of the surface elements in the image can be simplified further.

Furthermore, the surface elements may be, for example, triangular faces, preferably isosceles triangular faces, more preferably equilateral triangular faces, and/or the base body may be, for example, a polyhedron, preferably a regular polyhedron, more preferably a regular icosahedron.

Triangles are also mapped to triangles in a perspective image. Therefore, the identification of the surface elements in the image will also be simplified. In particular, triangles of the device are mapped to triangles in the image.

In another example, the base body can be an irregularly shaped body whose symmetry group comprises only the identity transformation.

For example, the third subset of the calibration objects may have no symmetry with respect to the base body under Euclidean spatial transformations, i.e. may completely break the symmetries of the base body.

This makes it easier to determine the orientation of the device from the image, or it makes the determination of a test calibration more reliable.

According to another example, the subsets of the calibration objects can differ from one another in shape and/or size and preferably be in the form of spheres.

This makes it easier to associate mappings of the calibration objects with the individual subsets.

The invention further relates to a computer-implemented method for calibrating a system for generating at least one image of an object by means of a device according to the preceding description, wherein each surface element has a shape, wherein object position data of the calibration objects in the device relative to one another are known and wherein the object position data of the calibration objects of the third subset relative to the object position data of the calibration objects of the first subset and the second subset differ between at least two surface elements, the method comprising the following steps: providing at least one image of the device by means of the system, the calibration objects in the at least one image being mapped to image calibration objects; determining image position data of all visible image calibration objects in the at least one image; identifying image surface elements of the device mapped in the at least one image by means of the determined image position data, the shapes of the surface elements and the object position data of at least the first subset and the second subset of the calibration objects, wherein image vertices and image outer edges are determined by means of the identified image surface elements; selecting a test calibration in which in a test mapping the object position data of the first subset of the calibration objects are mapped to the image vertices and the object position data of the second subset of the calibration objects are mapped to the image outer edges; determining test image position data of the third subset of the calibration objects by means of the test calibration; comparing the test image position data with the image position data of the third subset of the calibration objects; until the test image position data and the image position data of the third subset of the calibration objects are mapped to one another within a predefined tolerance range; repeating the steps of: selecting a modified test calibration in which in a test mapping the object position data of the first subset and the second subset of the calibration objects are mapped to the image vertices and the object position data of the second subset of the calibration objects in the image are mapped to the image outer edges, and determining test image position data of the third subset of the calibration objects and comparing the test image position data with the image position data of the third subset of the calibration objects with the modified test calibration.

In the inventive method, the device according to the above description is used for calibrating a system for generating at least one image of an object. The object position data of the calibration objects refer to the positioning of the calibration objects in the device. The object position data of the calibration objects relative to one another are known. Between the various surface elements of the device, the object position data of the calibration objects of the third subset relative to the object position data of the calibration objects of the first subset and the second subset differ. This means that the object position data of the calibration objects of the first and second subsets relative to one another can be the same for each surface element. However, the object position data of the calibration objects of the third subset relative to the object position data of the calibration objects of the first and second subsets differ for different, at least two, surface elements. For example, for a first surface element, a calibration object of the third subset can be arranged centrally between the calibration objects of the first and second subsets. For a second surface element, the corresponding calibration object of the third subset can be arranged e.g. off-center.

To calibrate the system, the computer-implemented method can first be used to generate at least one image of the device and to provide it, e.g. by means of a file on a storage medium, by way of data transmission from another computer or other methods or even directly. The system can be used to generate the at least one image. The calibration objects in the at least one image are mapped to image calibration objects. The term image calibration objects is used hereinbelow synonymously with the calibration objects of the device that are mapped in the image. The at least one image is usually a two-dimensional image, e.g. a projection representation or an optical representation of the device. However, this does not rule out the possibility of the at least one image being a three-dimensional image.

The image position data of the visible image calibration objects in the at least one image are then determined. To save computational effort, the image position data of the visible image calibration objects in a single image can first be determined, said data then also being used for the steps that follow. However, this does not preclude the image position data of the visible image calibration objects from also being determined in multiple or all provided images and used subsequently.

The image position data can be determined using known image analysis methods that are used to identify the image calibration objects in the image. Furthermore, the centers of the image calibration objects can be determined with high accuracy. If the calibration objects are e.g. spheres, they are mapped to ellipses in the image. The centers of the ellipses can then also be determined in order to estimate the position of the sphere centers in the image. The centers can then be assumed e.g. to be, if applicable, perspective image points of the three-dimensional calibration objects.

The steps that follow are used to associate the three-dimensional object position data of the calibration objects and the two-dimensional image position data of the image calibration objects with one another. This means that it is determined which image calibration object is the mapping of the corresponding calibration object from the device. The problem thus described and to be subsequently solved is referred to here and subsequently as the spherical correspondence problem.

For this purpose, the image surface elements in the image are identified, the determined image position data being used. As the relative relationships between the object position data are known among one another, this knowledge can be used to identify image surface elements in the image. If e.g. the surface elements have a triangular shape, with the calibration objects of the first subset arranged on the corners of the triangles and the calibration objects of the second subset arranged between the corners on the edges of the triangles, it is possible for e.g. three different image position data in the image to be checked for whether they satisfy these relative relationships for one another and for other image position data. If these relationships are satisfied, the three image position data are very likely arranged on the image vertices of an image surface element. If an image surface element is found, the image outer edges of the image surface elements adjacent to this image surface element are also known. On this basis, the identification of further image surface elements can be simplified. Simultaneously with the identification of the image surface elements, the image vertices and the image outer edges can thus also be identified.

If the image surface elements and thus the image vertices and image outer edges are known, the image calibration objects are associated with at least the three subsets of the calibration objects. The image calibration objects arranged on the image vertices are associated as mappings with the first subset of the calibration objects. The image calibration objects arranged on the image outer edges are associated as mappings with the second subset of the calibration objects. The remaining image calibration objects arranged within the image surface elements are associated as mappings with the third subset of the calibration objects.

A test calibration is then selected in which in a test mapping the object position data of the first or second subset are mapped to the image position data of the image calibration objects associated with the first or second subset. If the test calibration has been selected correctly, the correspondences between all known image calibration objects and corresponding calibration objects are known based on assumed correspondences between a few calibration objects and image calibration objects, e.g. when a single surface element is assumed to correspond to a particular image surface element. The selection of the test calibration may be an estimation that comprises the intrinsic and extrinsic detector parameters. The estimated detector parameters can then be used to perform the test mapping. The test mapping does not necessarily actually have to be performed, but can also be calculated or simulated. The calibration objects of the first and second subsets can thus be used to construct the test calibration, so that for the test calibration constructed in this way it is then checked whether the image position data of the image calibration objects can result from a mapping of object position data of corresponding calibration objects of the first or second subset that is produced by means of the test calibration. Since the test calibration is in a form such that at least some of the object position data of the calibration objects of the first and second subsets are mapped to some of the image position data of the corresponding image calibration objects, particularly the test image position data resulting from the object position data of the calibration objects of the third subset must, in principle, be determined. These are then compared with the image position data of the image calibration objects of the third subset.

If the test image position data do not match the image position data of the calibration objects of the third subset within a predefined tolerance range, the test calibration is not correct and is discarded. The predefined tolerance range can define a maximum permitted variance between the test image position data and the image position data of the calibration objects of the third subset. If the surface elements are triangles, the predefined tolerance range can be defined using barycentric triangle coordinates. A modified test calibration is selected, and the test by means of the test mapping outlined above is repeated, until the test image position data match the image position data of the calibration objects of the third subset within the predefined tolerance range.

In an alternative example of the method, all possible test calibrations are checked and then compared with one another to decide which test calibration is the most accurate.

When the test calibration has been determined in this way, the intrinsic and extrinsic detector parameters are sufficiently well known, and so the calibration of the system can be completed. Furthermore, at least a subset of object position data which corresponds to a subset of image position data is then known. This solves the spherical correspondence problem.

The invention thus provides a method for calibrating a system for generating at least one image of an object that can be used to determine the detector parameters with high accuracy with comparatively little computational effort.

According to one example, if the test image position data and the image position data of the third subset of the calibration objects are mapped to one another within a predefined tolerance range, the method can further comprise the following step: optimizing the test calibration by means of a nonlinear optimization method.

The nonlinear optimization method can be used to increase the accuracy of the test calibration, or calibration. In this instance, for the correspondences that are now known between the object position data and the image position data, adjusting the determined test calibration reduces the particular determined geometric error further and further.

In another example, the method can further comprise, before the step of: selecting a test calibration, the step of: determining the intrinsic detector parameters by means of at least the image position data of the image vertices and image outer edges, wherein the step of selecting a test calibration can comprise the following sub-step: determining extrinsic sample detector parameters by means of the determined intrinsic detector parameters and the image position data of an image surface element.

In this case, the intrinsic detector parameters are first determined before the extrinsic detector parameters are determined. This is advantageous particularly if the device has high symmetry. If the base body of the device is e.g. in the form of a regular icosahedron and the calibration objects of the first and second subsets are arranged identically for each surface element, the object position data of the calibration objects of the first subset form equilateral triangles. On the one hand, this simplifies the identification of the image surface elements. On the other hand, an overdetermined system of equations for the intrinsic detector parameters can be established by means of the determined image surface elements. This means that the intrinsic camera parameters can be determined or estimated with sufficient accuracy with little effort. For the test calibration, only the extrinsic detector parameters then need to be estimated as extrinsic sample detector parameters. The estimation of the extrinsic sample detector parameters can then be performed in this manner on the basis of the determined intrinsic detector parameters and the image position data of a single image surface element. In particular, it is advantageous that just a single image of the object can be used for determining the test calibration, instead of having to use multiple or all available images for determining the test calibration. That is to say that this method works even if the images are not recorded in a coherent trajectory, but only in an arbitrary sequence of images or even if only a single image is recorded. This is a major advantage especially when it is only possible to determine an image of the object from very specific positions, e.g. in the case of robot CT.

The determining of the intrinsic detector parameters and the determining of the extrinsic sample detector parameters can also be performed by estimating the intrinsic detector parameters or the extrinsic sample detector parameters.

For example, the method may further include the following step: performing a bundle adjustment method for all images for which intrinsic and extrinsic detector parameters have been determined.

The performance of a bundle adjustment method allows compensation for variances from the expected geometry of the device, i.e. slightly shifted object position data of the calibration objects in the device, which arise during the manufacture of the device in comparison with the target position data, or variances due to temperature expansion.

Further, the step of: determining the intrinsic detector parameters by means of at least the image position data of the image vertices and image outer edges can, for example, be carried out for all provided images before the step of: determining the extrinsic detector parameters for each surface element by means of the determined intrinsic detector parameters is carried out.

In this way, the intrinsic detector parameters are first optimized before the extrinsic detector parameters are optimized.

The invention further relates to the use of a device according to the preceding description for calibrating a system for generating at least one image of an object.

Advantages and effects and also developments of the use of the device result from the advantages and effects and also developments of the device described above and, if applicable, of the computer-implemented method described above. Reference is therefore made to the preceding description in this respect.

In another aspect, the invention relates to a computer program product comprising instructions that can be executed on a computer and that, when executed on a computer, cause the computer to perform the method according to the preceding description.

Advantages and effects and also developments of the computer program product result from the advantages and effects and also developments of the method described above. Reference is therefore made to the preceding description in this respect. A computer program product can be understood to mean e.g. a data storage medium which stores a computer program element comprising instructions which can be executed by a computer. Alternatively or in addition, a computer program product may also be understood as meaning, for example, a permanent or volatile data memory such as a flash memory or a main memory which has the computer program element. However, other types of data memories that comprise the computer program element are not excluded thereby.

Further features, details and advantages of the invention emerge from the wording of the claims and from the following description of exemplary embodiments on the basis of the drawings, in which:

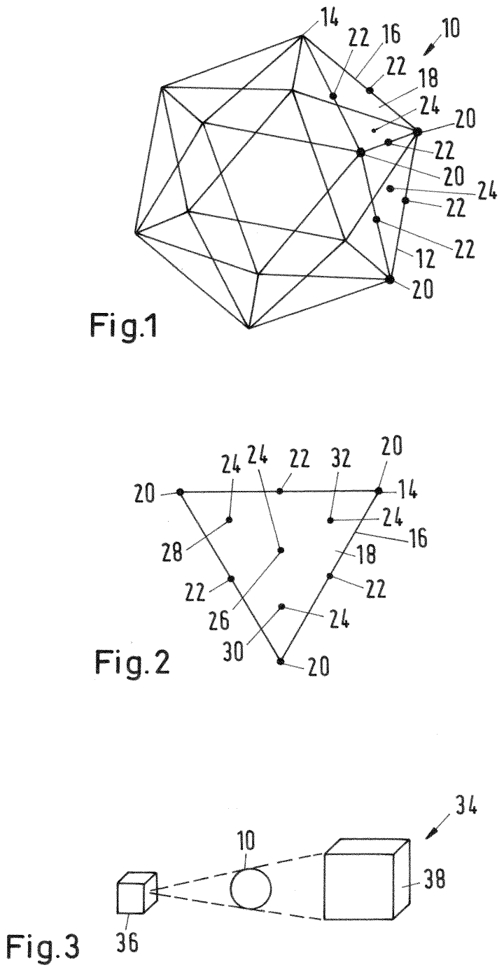

FIG. 1 shows a schematic representation of a base body of the device;

FIG. 2 shows a schematic representation of a surface element with calibration objects;

FIG. 3 shows a schematic representation of a system for generating at least one image of an object;

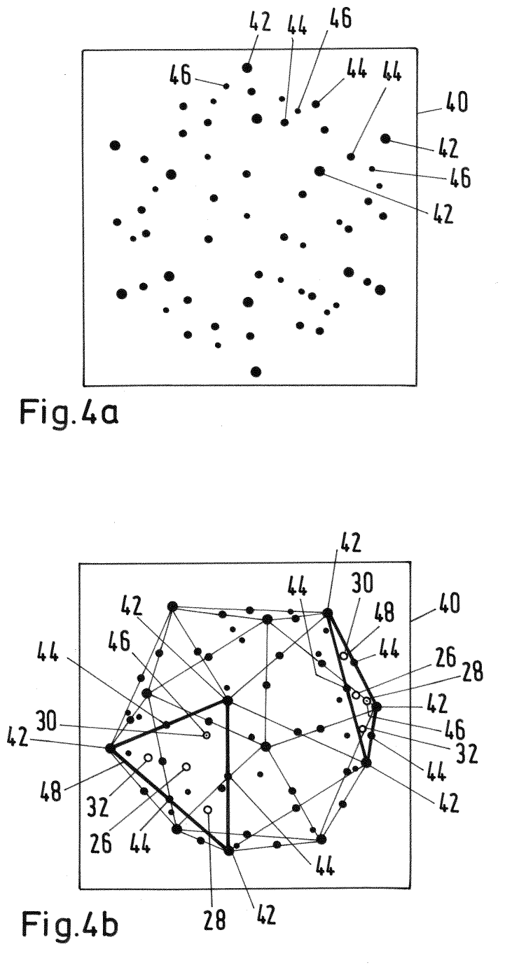

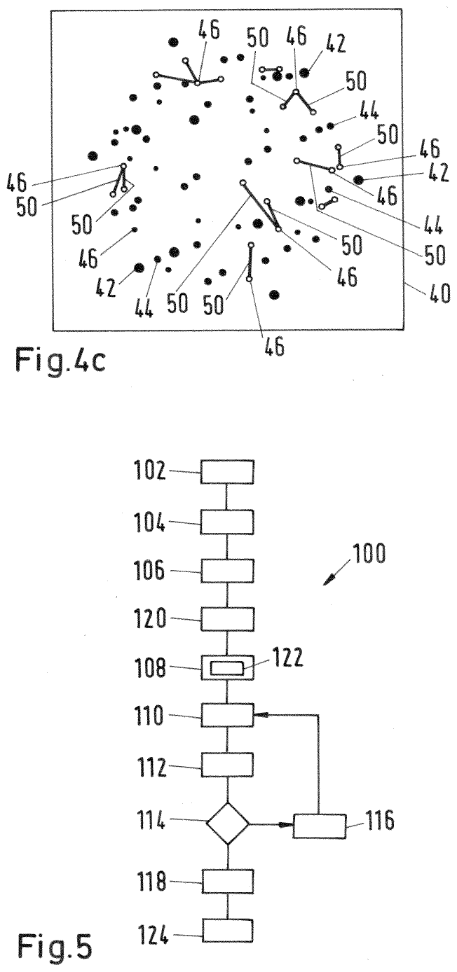

FIG. 4a-c show a schematic representation of an image of the device; and

FIG. 5 shows a graphical representation of the computer-implemented method.

Below, the device for calibrating a system for generating at least one image of an object in its entirety is referred to by the reference sign 10, as shown in FIG. 1.

The device 10 has a base body 12, which in this example is in the form of a regular icosahedron. The base body 12 has vertices 14, which form ends of straight outer edges 16. The outer edges 16 surround surface elements 18, which in this example are in the form of equilateral triangles. An example of a surface element 18 which is in the form of an equilateral triangle is shown in FIG. 2.

Furthermore, the device has a plurality of calibration objects 20, 22, 24. The calibration objects 20, 22, 24 can be in the form of spheres.

A first subset of the calibration objects is arranged on the vertices 14. These calibration objects are referred to by the reference sign 20.

A second subset of the calibration objects is arranged on the outer edges 16. These calibration objects are referred to by the reference sign 22. The calibration objects 22 of the second subset can be arranged centrally between the vertices 14 on the outer edges 16.

A third subset of the calibration objects is arranged on the surface elements 18. These calibration objects are referred to by the reference sign 24. In this example, the calibration objects 24 of the third subset can be arranged on the surface element 18 internally at one of four predefined positions 26-32. The remaining predefined positions 26-32 can be free of calibration objects 20, 22, 24. The predefined position 26 can be arranged at the center of the surface element 18. The predefined positions 28-32 may be arranged outside the center of the surface element 18 in a manner offset from a respective vertex 14 of the surface element 18.

The device 10 has at least two surface elements 18 for which the arrangement of the respective calibration object 24 of the third subset differs. That is to say, the calibration objects 24 of the third subset of the two surface elements 18 are arranged at positions different than the predefined positions.

Furthermore, a subset of the predefined positions 26-32 may be invariant among all symmetry transformations of the surface element 18. If e.g. the vertices 14 are permuted, the predefined positions 26-32 of the subset are transformed into one another.

In this example, in which the base body 12 is in the form of a regular icosahedron, all surface elements 18 have the same shape, which is shown in FIG. 2. The relative positions of the calibration objects 20 of the first subset and the calibration objects 22 of the second subset are then the same for all surface elements 18 of the base body 12. Only the relative positions of the calibration objects 24 of different surface elements 18 with respect to the calibration objects 20, 22 of the first and second subsets can differ from one another between the surface elements 18.

Under Euclidean spatial transformations, the calibration objects 24 of the third subset have no symmetries. This means that the calibration objects 24 of the third subset break the symmetry of the calibration objects or the base body 12.

In this example, in which the base body 12 is in the form of a regular icosahedron, four non-adjacent surface elements 18 can be selected in which the calibration objects 24 of the third subset are arranged in the position 26 at the center of the respective surface element 18. Each vertex 14 is then adjacent to exactly one surface element 18 for which a calibration object 24 of the third subset is arranged at the position 26.

For the remaining surface elements 18, the calibration objects 24 are arranged at each of the positions 28, 30, 32. The distribution over the positions 28, 30, 32 is produced in such a way that, for a revolution around a vertex 14 via the adjacent surface elements 18 starting from the surface element 18 for which a calibration object 24 of the third subset is arranged at the position 26, a unique distribution over the positions 28, 30, 32 arises with respect to the other vertices, regardless of the orientation that the respective distribution will be in.

It is particularly advantageous if the different distributions differ as clearly as possible.

The calibration objects 20, 22, 24 can differ from one another between the three subsets. The calibration objects 20, 22, 24 can differ from one another e.g. in their size or shape.

If the device 10 is designed for calibrating a system which generates images by means of radiographic measurements, the absorption properties of the calibration objects 20, 22, 24 differ from the absorption properties of the base body 12.

FIG. 3 shows a system 34 for generating at least one image of an object. In this example, the system 34 has a radiation source 36 and a detector 38. The radiation source 36 is present only in systems that perform a radiographic measurement. If the system 34 generates the image by means of optical radiation, the radiation source 36 can be omitted.

For calibrating the system 34, the device 10 is arranged such that the detector 38 can detect radiation that is reflected by the device 10 in the case of optical radiation and transmitted in the case of a radiographic measurement.

When a radiation source 36 is present, the radiation source 36 emits radiation in the direction of the device 10. The device 10 is then arranged between the radiation source 36 and the detector 38.

It is assumed below that the system 34 performs a radiographic measurement and can generate corresponding images of objects. However, the explanations that follow also apply to systems that generate images from radiation reflected from the surface.

For calibrating the system 34, the computer-implemented method 100 can be used to calibrate a system that is designed to generate at least one image of an object. The device 10 is used to perform the method 100. A flowchart for the method 100 is shown in FIG. 5.

According to step 102, the system 34 can be used to generate at least one image 40 of the device 10 and to provide it to the method 100, e.g. directly or by reading from a file previously stored on a storage medium. Examples of images 40 are shown in FIGS. 4a to 4c.

FIG. 4a shows an image 40 as the result of a radiographic measurement of the device 10. The image 40 comprises representations of the calibration objects 20, 22, 24 of the device, these representations being referred to below as image calibration objects 42, 44, 46. Since the calibration objects 20, 22, 24 are spherical, they were mapped as elliptical dark areas in the image 40. Initially, it is not known at which positions in the image 40 the image calibration objects 42, 44, 46 of the calibration objects 20, 22, 24 are arranged.

Therefore, in step 104, the image position data of the image calibration objects 42, 44, 46 visible in the image are determined. This can be done, for example, using known edge determination methods or the like.

The centers of the mapped elliptical areas of the image calibration objects 42, 44, 46 are preferably used as image position data.

In step 106, the image surface elements 48, as shown in FIG. 4b, are identified in the image 40 shown there. This image 40 may be the same image shown in FIG. 4a. In this example, multiple images were used and step 104 was carried out in all of them.

The image position data are used for step 106. Since the nature of the relative positional relationships of the calibration objects 20, 22, 24 in the device 10 is known, these relative positional relationships can also be used for identifying the image surface elements 48.

Identification of the image surface elements 48 can be simplified further and performed with greater certainty if the calibration objects 20, 22, 24 of the three subsets have different shapes and/or sizes. It is then possible, in principle, to directly associate the image calibration objects 42 which have the correct shape and/or size as mappings with the first subset of the calibration objects 20, direct association of the image calibration objects 42 corresponding to the calibration objects 20 not yet being possible at this juncture. The same applies to the calibration objects 22, 24. In order to group three image calibration objects 42 together with three image calibration objects 44 and an image calibration object 46 for each surface element 48 and thus identify the corresponding surface element in the image, the following procedure can be carried out: For all at all possible combinations of three (suspected) image calibration objects 42 and three (suspected) image calibration objects 44, it must first of all necessarily hold true that, within a tolerance range, the three (suspected) image calibration objects 44 are arranged relative to the three (suspected) image calibration objects 42 such that exactly one of the (suspected) image calibration objects 44 in each case is located on the connecting line between two of the (suspected) image calibration objects 42. If this is the case, the knowledge of the precise geometric shape of the surface element, i.e. the knowledge of the precise (known) relative positions of the calibration objects 20, 22 within the equilateral triangle, which are identical for each surface element, can be used to construct a so-called planar homography which reverses the perspective distortions created during the perspective mapping of the plane defined by the triangle.

Such a planar homography can be constructed as soon as at least four points are known that lie on a common plane. In this case, the six points defined by the selected image calibration objects 42, 44 are used. For all other image calibration objects for which it can be assumed, in principle, that, as image calibration object 46, they could potentially form a surface element 48 together with the three (suspected) image calibration objects 42 and the three (suspected) image calibration objects 44, the planar homography is now used to determine the location in the (suspected) equilateral triangle at which this (suspected) image calibration object 46 would be positioned. If the position thus determined is now at one of the positions 26, 28, 30, 32 within the (suspected) equilateral triangle within a tolerance range, then it is later (initially) assumed that the seven image calibration objects 42, 44, 46 grouped in this way are associated with an image surface element 48. By identifying the image surface element 48 by way of the association of seven image calibration objects 42, 44, 46 with known geometric positions (corners, edges and area) described here, it has now also been determined in every case which of the seven image calibration objects are associated with the calibration objects 20, 22, 24 of the first, second and third subsets if this was not able to be determined previously, e.g. using the different sizes and/or shapes of the calibration objects 20, 22, 24. This knowledge can be used in particular to reverse perspective distortions of the detector or camera at this common level.

The optional step 120 can now be used to determine the intrinsic detector parameters. This is accomplished by using the image position data of the image vertices and image outer edges of the image surface elements 48 on which the image calibration objects 42, 44 are arranged. The determination can also be referred to as an estimation of the intrinsic detector parameters.

For step 120, the knowledge of the planar homographies can be used. A known metric planar homography, as was determined here for each image surface element, results in two linear conditions for the components of the image of the absolute conic (IAC) for each of these planes. This allows an overdetermined linear system of equations to be established. This system of equations can be solved e.g. using a, if applicable linear, least squares method. Thereafter, e.g. a Cholesky factorization of the IAC can be used to obtain the intrinsic detector parameters. This can be done before it is known which calibration object 20, 22, 24 is mapped to which image calibration object 42, 44, 46.

In step 108 of the method 100, a test calibration is selected. The test calibration is selected by assuming corresponding calibration objects 20, 22, 24 for some image calibration objects 42, 44, 46, said corresponding calibration objects being mapped to these image calibration objects 42, 44, 46. This test calibration is then used to determine corresponding calibration objects 20, 22, 24 for further determined, preferably all determined, image calibration objects 42, 44, 46, and this association is logged or stored.

If step 120 has not been performed, multiple surface elements 18, at least two, preferably three, for which correspondences between the calibration objects 20, 22, 46 and the image calibration objects 42, 44, 24 are determined are selected in step 108, the correspondences for the remaining surface elements 18 then being determined by means of the selected test calibration. The determination of the test mapping only implicitly defines the extrinsic and intrinsic detector parameters in this case. At least one condition for determining the correspondences is that the calibration objects 20 of the first subset are mapped to the image calibration objects 42 and the calibration objects 22 of the second subset are mapped to the image calibration objects 44.

If, in an alternative example, the optional step 120 has been performed, the test calibration can be selected by means of a single surface element 18 for which correspondences between the calibration objects and the image calibration objects are determined. Furthermore, the optional sub-step 122 can then be carried out, in which case only the extrinsic detector parameters need to be selected for the test calibration in step 108, since the intrinsic detector parameters are then already known from carrying out step 120.

The optional step 120 may also be an optional sub-step of step 108 in one example, which is not shown.

In sub-step 122, the extrinsic detector parameters of a test calibration can be determined e.g. using a perspective-n-point method. For each image surface element determined in the image, it is possible to guess which of the surface elements has been mapped here. In addition, the permutation of the calibration objects of the first subset must also be guessed. Taking the example of an equilateral triangle as a surface element, there are a maximum of six possible permutations depending on the positioning of the calibration object of the third subset. There are only a small number of mapping options for a particular surface element. For each of these options, the intrinsic detector parameters and the correspondences between the image position data and the object position data that result from this selection can then be used to determine the extrinsic detector parameters using the perspective-n-point method, although all three-dimensional object position data lie in a common plane. A set of the extrinsic detector parameters determined in this way can define a test calibration in combination with the intrinsic detector parameters. For the test calibrations determined in this way, the calibration objects of the first and second subsets are generally mapped to the image position data on which the image calibration objects which are mappings of the first and second subsets are arranged.

A test calibration is then selected which is then used in step 110 to generate a test mapping for the device 10, this test mapping being able to be simulated or calculated without performing an actual mapping. In the test mapping, the test image position data of the mappings of the calibration objects 24 of the third subset are determined.

The test image position data are compared in step 112 with the image position data of the calibration objects 46 of the third subset. This comparison is illustrated in FIG. 4c. If the test calibration is correctly assumed and sufficiently accurate, the test image position data do not deviate from the image position data within a predefined tolerance range. However, if the test calibration is assumed incorrectly, there will be variances 50 between the test image position data and the image position data. The variances 50 resulting from an inaccurate test calibration are graphically represented in FIG. 4c as lines starting from the applicable image position data in FIG. 4c.

If the comparison from step 112 does not result in a sufficiently accurate test calibration, i.e. if the test image position data and the image position data of the third subset of the calibration objects are not mapped to one another within a predefined tolerance range, steps 116, 110 and 112 are repeated in step 114. The test calibration originally used is then discarded, as are logged correspondences between the image calibration objects 42, 44, 46 and the calibration objects 20, 22, 24. In step 116, a modified test calibration is selected, which, in a test mapping, maps the object position data of the first subset and the second subset of the calibration objects to the image vertices and the object position data of the second subset of the calibration objects to the image outer edges in the mapping. Step 116 is identical to step 108 except for the use of a modified test calibration. Therefore, steps 110 and 112 are carried out with the modified test calibration in the repetition.

If the optional sub-step 122 has been carried out, the test calibration determined to be inaccurate is discarded in the repetition and a previously unchecked set of extrinsic detector parameters is linked to the intrinsic detector parameters to form a modified test calibration.

If the comparison from step 112 results in a sufficiently accurate test calibration, i.e. if the test image position data and the image position data of the third subset of the calibration objects are mapped to one another within a predefined tolerance range, the method can be continued with the optional step 118. Furthermore, correspondences between the object position data and the image position data are then known.

In an alternative example of the method, all possible test calibrations are checked and then compared with one another to decide which test calibration is the most accurate.

In the optional step 118, the accuracy of the determined sufficiently accurate test calibration can be increased by means of a nonlinear optimization method. In addition, with the sufficiently accurate test calibration, an estimation, a so-called initial guess, of the intrinsic and extrinsic detector parameters is now known which also takes special mapping properties of the detector, in particular square and non-affinely distorted pixels, into account at least approximately. Using such an initial guess and the correspondences determined in this way, the nonlinear optimization method can be used to adapt the intrinsic and extrinsic detector parameters in such a way that the so-called geometric error of the determined correspondences between the image position data and the object position data is minimized.

If the optional step 120 has not been performed, the optional step 118 optimizes a mapping matrix to improve the implicitly defined detector parameters. If the optional step 120 has been performed, step 118 optimizes the detector parameters. The special mapping properties of the detector, in particular square and non-affinely distorted pixels, are taken into account exactly, since then the intrinsic detector parameters are immediately optimized further. The nonlinear optimization from step 118 then takes into account the special mapping properties of the detector, i.e. the intrinsic detector parameters determined from step 120, which already take these special mapping properties of the detector into account exactly. After the optimization by step 118, the abovementioned special mapping properties continue to also apply exactly to the optimized test calibration.

The steps of the method 100 that have been described above can advantageously be carried out only using a single image of the device. Alternatively, the steps of the method 100 that have been described above can be carried out with multiple images of the device, the device preferably being mapped, in each of the images used, under a different orientation with respect to the detector or the camera of the system.

In a further optional step 124, a bundle adjustment method can be performed for all images of the device that have been generated using the system. The bundle adjustment method can e.g. compensate for slight shifts in the object position data of the calibration objects in comparison with target position data of the calibration objects in the device.

In this case, a nonlinear optimization method can optimize the detector parameters of all images and also the object position data of the calibration objects together in order to minimize the reprojection error of the correspondences found between the image position data and the object position data across all images. The previously determined detector parameters and also the known target positions of the calibration objects serve as an initial guess for the nonlinear optimization.

The invention is not restricted to one of the embodiments described above, but rather can be modified in various ways. All of the features and advantages emerging from the claims, the description and the drawing, including design details, spatial arrangements and method steps, can be essential to the invention both alone and in a wide variety of combinations.

1. A device for calibrating a system for generating at least one image of an object, wherein the device comprises a plurality of calibration objects and a base body having vertices and outer edges that extend between the vertices and delimit surface elements, wherein a first subset of the calibration objects is arranged on the vertices of the base body, and a second subset of the calibration objects is arranged on the outer edges between the vertices, and a third subset of the calibration objects is arranged between the outer edges on the surface elements.

2. The device as claimed in claim 1, wherein the arrangement of the calibration objects of the third subset relative to the calibration objects of the first subset and the second subset differs between at least two surface elements.

3. The device as claimed in claim 1, wherein each outer edge has at most one associated calibration object of the second subset of the calibration objects, and/or the second subset of the calibration objects is arranged centrally between the vertices on each outer edge.

4. The device as claimed in claim 1, wherein each surface element has at least two, predefined positions internally, wherein each surface element has an associated calibration object of the third subset of the calibration objects, which calibration object is arranged at one of the predefined positions and the other predefined positions are free of calibration objects wherein more preferably a subset of the predefined positions is invariant under all symmetry transformations of the surface element.

5. The device as claimed in claim 1, wherein the relative arrangement of the calibration objects of the first subset and the second subset in relation to one another on each surface element is the same.

6. The device as claimed in claim 1, wherein the surface elements are triangular faces and/or the base body is a polyhedron.

7. The device as claimed in claim 1, wherein the third subset of the calibration objects has no symmetry with respect to the base body under Euclidean spatial transformations.

8. The device as claimed in claim 1, wherein the subsets of the calibration objects differ from one another in shape and/or size and are in the form of spheres.

9. A computer-implemented method for calibrating a system for generating at least one image of an object by means of a device as claimed in claim 1, wherein each surface element has a shape, wherein object position data of the calibration objects in the device relative to one another are known and wherein the object position data of the calibration objects of the third subset relative to the object position data of the calibration objects of the first subset and the second subset differ between at least two surface elements, the method comprising the following steps:
providing at least one image of the device by means of the system, the calibration objects in the at least one image being mapped to image calibration objects;
determining image position data of all visible image calibration objects in the at least one image;
identifying image surface elements of the device mapped in the at least one image by means of the determined image position data, the shapes of the surface elements and the object position data of at least the first subset and the second subset of the calibration objects, wherein image vertices and image outer edges are determined by means of the identified image surface elements;
selecting a test calibration in which in a test mapping the object position data of the first subset of the calibration objects are mapped to the image vertices and the object position data of the second subset of the calibration objects are mapped to the image outer edges;
determining test image position data of the third subset of the calibration objects by means of the test calibration;
comparing the test image position data with the image position data of the third subset of the calibration objects;
until the test image position data and the image position data of the third subset of the calibration objects are mapped to one another within a predefined tolerance range: repeating the steps of:
selecting modified test calibration in which in a test mapping the object position data of the first subset and the second subset of the calibration objects are mapped to the image vertices and the object position data of the second subset of the calibration objects in the image are mapped to the image outer edges, and
determining test image position data of the third subset of the calibration objects and comparing the test image position data with the image position data of the third subset of the calibration objects with the modified test calibration.

10. The method as claimed in claim 9, wherein when the test image position data and the image position data of the third subset of the calibration objects are mapped to one another within a predefined tolerance range, the method further comprises the following step:
optimizing the test calibration by means of a nonlinear optimization method.

11. The method as claimed in claim 9, wherein the method further comprises, before the step of: selecting a test calibration, the step of:
determining the intrinsic detector parameters by means of at least the image position data of the image vertices and image outer edges;
wherein the step of: selecting a test calibration comprises the following sub-step:
determining extrinsic sample detector parameters by means of the determined intrinsic detector parameters and the image position data of an image surface element.

12. The method as claimed in claim 11, wherein the method further comprises the following step:
performing a bundle adjustment method for all images for which intrinsic and extrinsic detector parameters have been determined.

13. The method as claimed in claim 11, wherein the step of: determining the intrinsic detector parameters by means of at least the image position data of the image vertices and image outer edges is carried out for all provided images before the step of: determining the extrinsic detector parameters for each surface element by means of the determined intrinsic detector parameters is carried out.

14. The use of a device as claimed in claim 1, for calibrating a system for generating at least one image of an object or for solving a spherical correspondence problem.

15. A computer program product comprising instructions that can be executed on a computer and that, when executed on a computer, cause the computer to perform the method as claimed in claim 1.
